(19) United States
(12) Patent Application Publication
(10) Pub. No.: US 2012/0071760 A1
(43) Pub. Date: Mar. 22, 2012

(54) RECONFIGURABLE MEDICAL ULTRASOUND TRANSDUCER ARRAYS WITH PREPROCESSING

(75) Inventors: Gregg MILLER, BOULDER, CO (US); Chris SANDERS, Redmond, WA (US)

(73) Assignees: TOSHIBA MEDICAL SYSTEMS CORPORATION, OTAWARA-SHI (JP); KABUSHIKI KAISHA TOSHIBA, TOKYO (JP)

(21) Appl. No.: 12/888,276

(22) Filed: Sep. 22, 2010

(51) Int. Cl. A61B 8/14

(52) U.S. Cl. 600/443

(57) ABSTRACT

The embodiments of the probe include a preprocessing unit that is directly connected to a first predetermined number of the receiving elements for preprocessing the channel signals to generate preprocessed channel signals as well as a switch unit that is connected to the preprocessing unit for connecting the preprocessed channel signals into a predetermined number of output channel signals. In the above embodiments, the second predetermined number is smaller than the first predetermined number. In addition, the embodiments of the probe also include an analog-to-digital converter connected to the switch unit for converting the output channel signals into digital signals, and a dynamic range of the analog-to-digital converter is related to a ratio between the second predetermined number and the first predetermined number.

### FIELD

Embodiments described herein relate generally to ultrasound diagnostic imaging systems and method of operating the same.

### BACKGROUND

As illustrated in FIG. 1, a conventional ultrasound imaging system includes a system unit 10, a cable 30 and an ultrasound probe handle 40. The probe 40 is connected to the system 10 via the cable 30. The system unit 10 generally controls a transducer 41 such as a 2-dimensional array in the probe handle 40 for transmitting ultrasound pulses towards a region of interest in a patient and receiving the ultrasound echoes reflected from the patient. The system unit 10 concurrently receives in real time the reflected ultrasound signals or echoes for further processing so as to display on a display unit an image of the region of the interest.

In detail, the probe 40 further includes the 2-dimensional transducer array 41 having a predetermined number of transducer elements, which are grouped into channels for transmitting the ultrasound pulses and receiving the ultrasound echoes. For 2-dimensional (2D) imaging data, a number of channels ranges from 64 to 256. On the other hand, for 3-dimensional (3D) imaging data, a number of required channels often exceeds 1000's. In the above described conventional ultrasound imaging system, the transducer unit 40 sends the system unit 10 via the cable 30 a large volume of reflected ultrasound data for real-time imaging.

In general, it is desired to shorten a time period of 4D ultrasound examinations in order to maximize the clinical utilization of ultrasound imaging systems. Even highly skilled sonographers occupy the ultrasound system and require the patient to be present for a relatively long period of time in comparison to some other imaging modalities. In 2D imaging, and particular 3D/4D imaging, these concerns and desires have led to certain prior art attempts.

To have an improved or efficient workflow of the ultrasound examination, patients are examined using an ultrasound imaging device as quickly as possible to acquire data, and the stored diagnostic images are manipulated and analyzed generally after the patients have already left the examination sessions. Particularly, in the advent of volumetric imaging or also known as sonographic tomography, a single sweep of a region using modern 2D array ultrasound may provide all necessary data for later analyses. To ensure that the transducer is positioned to capture a diagnostically relevant volume and that the system parameters are appropriately set for meaningful data, a conventional 2D image is provided as a good acoustic window to the operator before initiating the diagnostically relevant 3D/4D data. Ultimately, the improved workflow enhances the utilization of the imaging devices.

At the same time, the image quality needs to be maintained for diagnostic accuracy while the above described workflow of the ultrasound examination is improved. In this regard, the received signals undergo improved signal processing for the efficient workflow of the ultrasound examination using a reconfigurable transducer array.

### BRIEF DESCRIPTION OF THE DRAWINGS

FIG. 1 is a diagram illustrating units of one exemplary prior art ultrasound imaging system.

FIG. 2 is a diagram illustrating components of receive electronics in one exemplary prior art ultrasound imaging system.

FIG. 3 is a diagram illustrating an embodiment of the probe according to the current invention.

FIG. 4 is a diagram illustrating further detail of the first embodiment of the probe according to the current invention.

FIG. 5 is a diagram illustrating further detail of the second embodiment of the probe 200 according to the current invention.

FIG. 6 is a diagram illustrating further detail of the third embodiment of the probe 300 according to the current invention.

FIG. 7 is a diagram illustrating one embodiment of a two-dimensional array of transducer elements in the probe according to the current invention.

FIG. 8 is a diagram illustrating a first exemplary pattern of a two-dimensional array of transducer elements in a predetermined survey mode according to the current invention.

FIG. 9 is a diagram illustrating a second exemplary pattern of a two-dimensional array of transducer elements in a predetermined survey mode according to the current invention.

FIG. 10 is a diagram illustrating a third exemplary pattern of a two-dimensional array of transducer elements in a predetermined survey mode according to the current invention.

FIGS. 11A and 11B are diagrams illustrating further detail of the first embodiment of the probe according to the current invention.

FIG. 12 is a flow chart illustrating the steps or acts involved in one embodied process of operation with respect to the use of the probe according to the current invention.

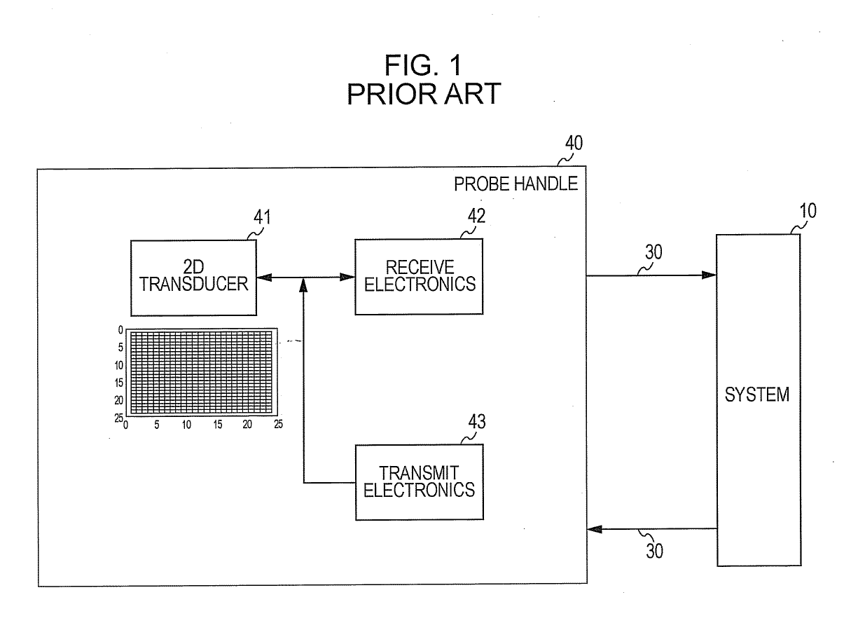

### DETAILED DESCRIPTION

Embodiments of the ultrasound imaging system according to the current invention include a probe or transducer unit, a processing unit and a cable connecting the probe to the processing unit. In general, the embodiments of the probe include some structures, components and elements of a conventional ultrasound probe. That is, one embodiment of the probe generates ultrasound pulses and transmits them towards a certain area of a patient. The embodiment also receives the ultrasound echoes reflected from the patient while many embodiments of the probe are generally hand-held devices, some are not hand-held devices.

The embodiments of the probe additionally include at least one preprocessing unit for processing 2D and or 3D/4D imaging data that is received at the probe. The term, “preprocessing” unit is synonymous with devices or circuits for further processing input signals that are directly inputted to the preprocessing unit from the receive elements of the array and is used to distinguish the “processing” for further processing input signals that are not directly inputted to the processing unit from the receive elements of the array. In other words, the preprocessing unit is connected to the receive elements of the transducer without any other electronic components such as cross point switches and or multiplexers (MUX) so that original input signals are maintained from the channels representing individual receive elements.

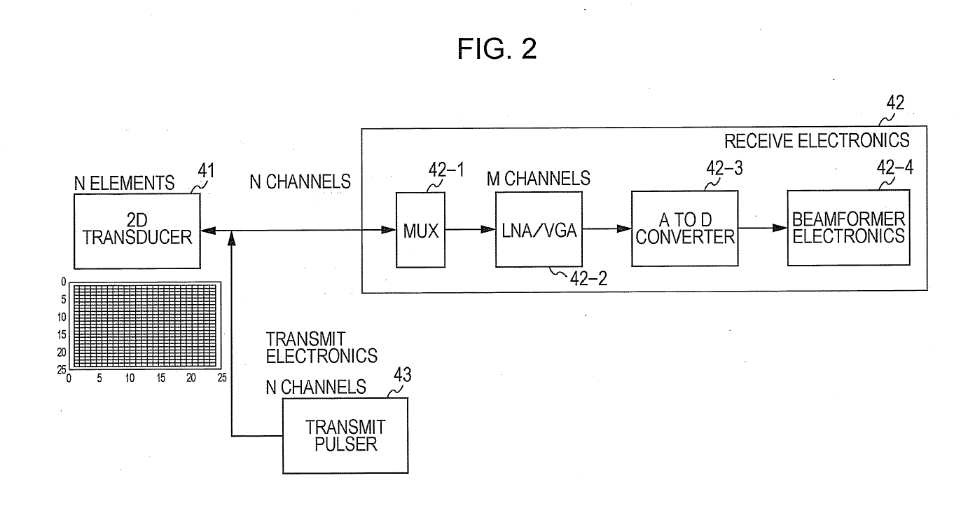

To illustrate the above described signal processing, FIG. 2 illustrates certain components of a prior art probe. An exemplary prior art probe includes a 2D transducer 41 having N elements and a transmit electronics such as a transmit pulser or a transmitting unit 43 for transmitting N channels of ultrasound pulses. The exemplary prior art probe also includes a receive electronics 42 that includes a multiplexer (MUX) 42-1 for connecting the N input channels together such that the number of M output channels is less than the number of the N input channels. The signal from the reduced number of M output channels is subsequently processed. In this regard, the receive electronics 42 further includes a post-MUX processing unit 42-2, which performs low noise amplifier (LNA) and or voltage gain amplification (VGA) on the signals from the M output channels. The processed analog signal is converted to digital signal via an analog-to-digital signal converter (ADC) 42-3, and the digital signal is inputted to a beamformer electronics 42-4.

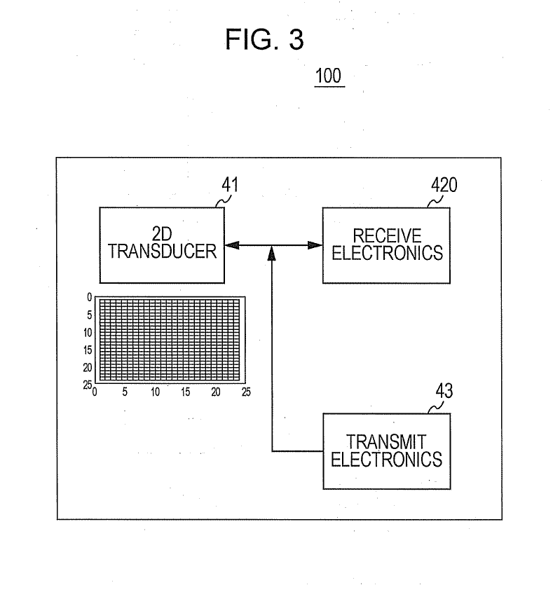

Referring now to the drawings, wherein like reference numerals designate corresponding structures throughout the views, and referring in particular to FIG. 3, a diagram illustrates a first embodiment of the probe 100 according to the current invention. In general, the first embodiment of the probe according to the current invention also generally includes a 2D transducer 41, a transmit electronics 43 and a receive electronics 420. The 2D transducer 41 is connected to both the transmit electronics 43 and the receive electronics 420 so that the 2D transducer 41 transmits ultrasound pulses from transmit elements towards a region of interest of a subject and receive ultrasound echoes that have been reflected from the region of interest of the subject at a first predetermined number of receiving elements of the transducer array so that the ultrasound echoes are converted into the first predetermined number of channel signals. Although the current invention is not limited, the number of the transmit elements and the receive elements are generally the same. Furthermore, the current invention is also not limited to the use of a two-dimensional transducer array.

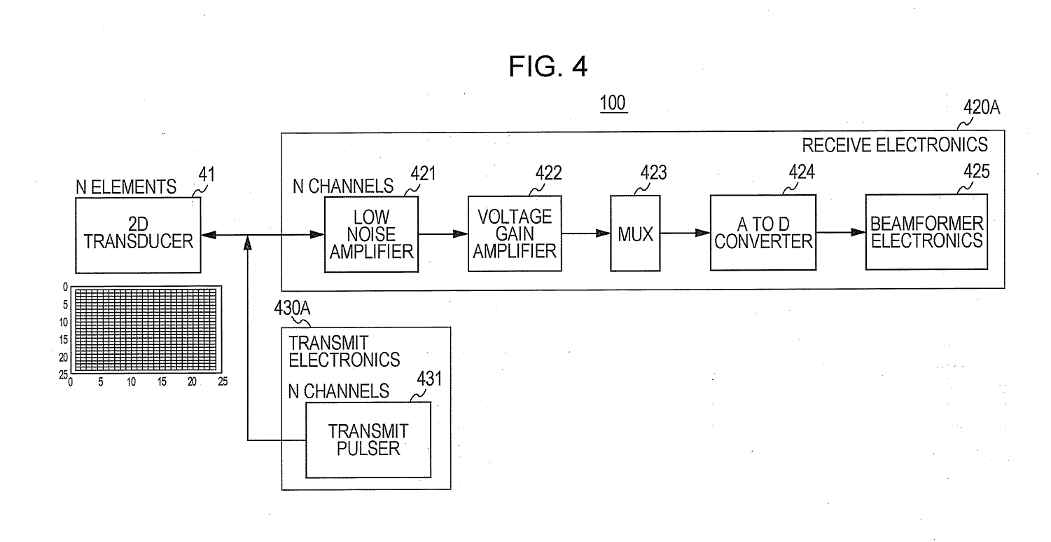

Now referring in particular to FIG. 4, a diagram illustrates further detail of the first embodiment of the probe 100 according to the current invention. The first embodiment of the probe 100 includes a 2D transducer 41, the transmit electronics 430A and a receive electronics 420A, which is a particular example of the receive electronics 420 of FIG. 3. The 2D transducer 41 is connected to both the transmit electronics 430A and the receive electronics 420A so that the 2D transducer 41 transmits ultrasound pulses from transmit elements towards a region of interest of a subject and receive ultrasound echoes that have been reflected from the region of interest of the subject at a first predetermined number of receiving elements of the transducer array so that the ultrasound echoes are converted into the first predetermined number of channel signals. Although the current invention is not limited, the number of the transmit elements and the receive elements are generally the same as already mentioned. Furthermore, the current invention is also not limited to the use of a two-dimensional transducer array as also already mentioned.

Still referring to FIG. 4, the receive electronics 420A performs a predetermined sequence of signal processing on the input from the 2D transducer 41. The 2D transducer 41 utilizes a predetermined number of N receive elements, which output N number of channel signals. In certain situation, the 2D transducer 41 optionally utilizes less than all of the available receive elements. Thus, the receive electronics 420A receives the N-channel signals from the 2D transducer 41 to via connections as indicated by arrows. In this exemplary embodiment, the receive electronics 420A includes a processing unit which is a combination of devices such as a low noise amplifier (LNA) 421 and a voltage gain amplifier 422. In addition, the receive electronics 420A in the exemplary embodiment includes a multiplexer (MUX) 423, an analog-to-digital signal converter (ADC) 424 and beamformer electronics 425. In this particular exemplary embodiment, the low noise amplifier (LNA) 421 initially performs low noise amplification on the N-channel signals directly received from the 2D transducer 41. In other words, there is no switching or any other processing on the N-channel signals between the 2D transducer 41 and the LNA 421. The LNA 421 amplifies signals while it matches input impedance to element impedance for maximum signal-to-noise ratio and bandwidth. Subsequently, the voltage gain amplifier (VGA) 422 performs voltage gain amplification on the output from the LNA 421 in this particular exemplary embodiment. The VGA 422 amplifies signals while it gains changes with time and or depth.

After every one of the signals from the 2D transducer 41 has been pre-processed by the LNA 421 and the VGA 422, the multiplexer (MUX) 423 connects channels together such that the number of output channels is less than the number of input channels to the MUX 423. In other words, the MUX 423 multiplexes only the pre-processed channel signals in the exemplary embodiment of the current invention. The above connecting or switching operation is reconfigurable in selecting a combination of the reduced number of channels and the positions of the corresponding receiving elements in the transducer array for the preprocessed channel signals. As a result of the multiplexing in the receive electronics 420A, some of the original signals from the N received elements are now combined into the M channel signals, which is less than the predetermined number N. The above connecting or switching operation is performed for optimizing signal fidelity along an ultrasound beam direction of interest. Although the exemplary embodiment utilizes a multiplexer, another type of switching unit such as a cross point switch is optionally utilized to practice the current invention.

After multiplexing process, the receive electronics 420A of the exemplary embodiment further performs additional signal processing within a probe handle 100. The multiplexed M channel signal now undergoes analog-to-digital signal conversion at the analog-to-digital signal converter (ADC) 424. A resolution level of the analog-to-digital conversion is related to a ratio between a reduced number of output channels and an original number of input channels in the connecting or switching operation. After the beamformer electronics 425 receives the converted digital signal from the ADC 424, the beamformer electronics 425 delays and sums the digital signals in order to form a coherent ultrasound beam.

In above exemplary embodiment, the receive electronics 420A includes the low noise amplifier (LNA) 421, the voltage gain amplifier (VGA) 422, the multiplexer (MUX) 423, the analog-to-digital signal converter (ADC) 424 and the beamformer electronics 425. Since these components are illustrative, other embodiments are not necessarily limited to a combination of the components and a particular sequence of the components in order to practice the current invention. Furthermore, other embodiments are not necessarily limited to a combination of the components that are located in the probe in order to practice the current invention.

Still referring to FIG. 4, the first embodiment of the probe 100 also includes the transmit electronics 430A, which in turn further includes a transmit pulser 431. As an arrow from the transmit electronics 430A indicates, the transmit pulser 431 controls the generation of ultrasound pulses from transmit elements through a predetermined number of N channels in the 2D transducer 41. In the exemplary embodiment of the probe 100, the transmit electronics 430A performs substantially no preprocessing that is equivalent or related to the above discussed preprocessing of the receive electronics 420A. On the other hand, in an alternative embodiment of the probe 100, the transmit electronics 430A performs some preprocessing function such as low noise amplification by placing a low noise amplifier in a transmitter chip.

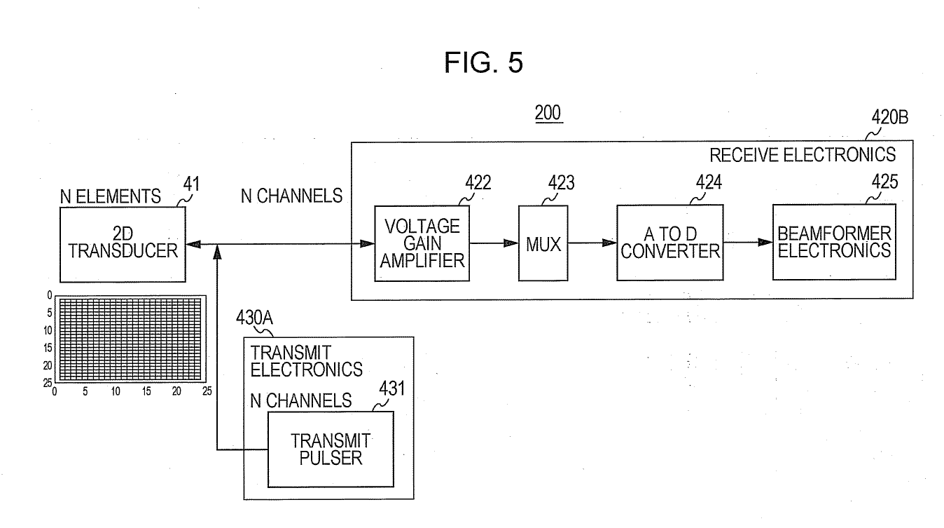

Now referring in particular to FIG. 5, a diagram illustrates further detail of the second embodiment of the probe 200 according to the current invention. The second embodiment of the probe 200 includes a 2D transducer 41, the transmit electronics 430A and a receive electronics 420B, which is a particular example of the receive electronics 420 of FIG. 3. The 2D transducer 41 is connected to both the transmit electronics 430A and the receive electronics 420B so that the 2D transducer 41 transmits ultrasound pulses from transmit elements towards a region of interest of a subject and receive ultrasound echoes that have been reflected from the region of interest of the subject at a first predetermined number of receiving elements of the transducer array so that the ultrasound echoes are converted into the first predetermined number of channel signals. Although the current invention is not limited, the number of the transmit elements and the receive elements are generally the same as already mentioned. Furthermore, the current invention is also not limited to the use of a two-dimensional transducer array as also already mentioned.

Still referring to FIG. 5, the receive electronics 420B performs a predetermined sequence of signal processing on the input from the 2D transducer 41. The 2D transducer 41 utilizes a predetermined number of N receive elements, which output N number of channel signals. In certain situation, the 2D transducer 41 optionally utilizes less than all of the available receive elements. Thus, the receive electronics 420B receives the N-channel signals from the 2D transducer 41 to via connections as indicated by arrows. In this exemplary embodiment, the receive electronics 420B includes a voltage gain amplifier 422, a multiplexer (MUX) 423, an analog-to-digital signal converter (ADC) 424 and beamformer electronics 425. In this particular exemplary embodiment, the voltage gain amplifier (VGA) 422 initially performs voltage gain amplification on the N-channel signals directly received from the 2D transducer 41 in this particular exemplary embodiment. The VGA 422 amplifies signals while it gains changes with time and or depth. In other words, there is no switching or any other processing on the N-channel signals between the 2D transducer 41 and the VGA 422.

After every one of the signals from the 2D transducer 41 has been pre-processed only by the VGA 422, the multiplexer (MUX) 423 connects channels together such that the number of output channels is less than the number of input channels to the MUX 423. In other words, the MUX 423 multiplexes only the pre-processed channel signals in the exemplary embodiment of the current invention. The above connecting or switching operation is reconfigurable in selecting a combination of the reduced number of channels and the positions of the corresponding receiving elements in the transducer array for the preprocessed channel signals. As a result of the multiplexing in the receive electronics 420B, some of the original signals from the N received elements are now combined into the M channel signals, which is less than the predetermined number N. The above connecting or switching operation is performed for optimizing signal fidelity along an ultrasound beam direction of interest. Although the exemplary embodiment utilizes a multiplexer, another type of switching unit such as a cross point switch is optionally utilized to practice the current invention.

After multiplexing process, the receive electronics 420B of the exemplary embodiment further performs additional signal processing within a probe handle 200. The multiplexed M channel signal now undergoes analog-to-digital signal conversion at the analog-to-digital signal converter (ADC) 424. A resolution level of the analog-to-digital conversion is related to a ratio between a reduced number of output channels and an original number of input channels in the connecting or switching operation. After the beamformer electronics 425 receives the converted digital signal from the ADC 424, the beamformer electronics 425 delays and sums the digital signals in order to form a coherent ultrasound beam.

In above exemplary embodiment, the receive electronics 420B includes the voltage gain amplifier (VGA) 422, the multiplexer (MUX) 423, the analog-to-digital signal converter (ADC) 424 and the beamformer electronics 425. Since these components are illustrative, other embodiments are not necessarily limited to a combination of the components and a particular sequence of the components in order to practice the current invention. For example, a low noise amplifier optionally replaces the VGA 422 in an alternative embodiment. Furthermore, other alternative embodiments are not necessarily limited to a combination of the components that are located in the probe in order to practice the current invention.

Still referring to FIG. 5, the second embodiment of the probe 200 also includes the transmit electronics 430A, which in turn further includes a transmit pulser 431. As an arrow from the transmit electronics 430A indicates, the transmit pulser 431 controls the generation of ultrasound pulses from transmit elements through a predetermined number of N channels in the 2D transducer 41. In the exemplary embodiment of the probe 200, the transmit electronics 430A performs substantially no preprocessing that is equivalent or related to the above discussed preprocessing of the receive electronics 420B.

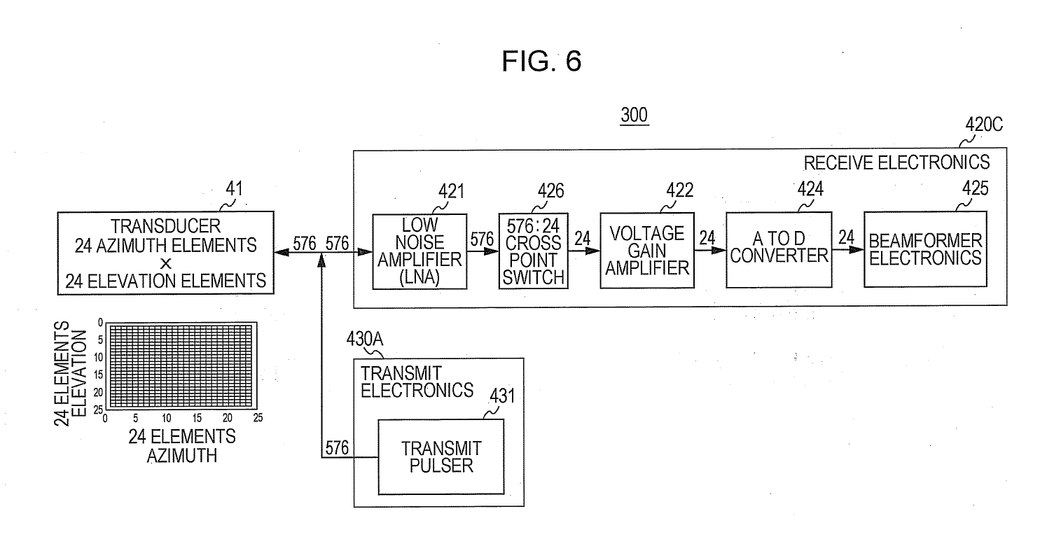

Now referring in particular to FIG. 6, a diagram illustrates further detail of the third embodiment of the probe 300 according to the current invention. The third embodiment of the probe 300 includes a 2D transducer 41, the transmit electronics 430A and a receive electronics 420C, which is a particular example of the receive electronics 420 of FIG. 3. The 2D transducer 41 is connected to both the transmit electronics 430A and the receive electronics 420C so that the 2D transducer 41 transmits ultrasound pulses from transmit elements towards a region of interest of a subject and receive ultrasound echoes that have been reflected from the region of interest of the subject at a first predetermined number of receiving elements of the transducer array so that the ultrasound echoes are converted into the first predetermined number of channel signals. Although the current invention is not limited, the number of the transmit elements and the receive elements are generally the same as already mentioned. Furthermore, the current invention is also not limited to the use of a two-dimensional transducer array as also already mentioned.

Still referring to FIG. 6, the receive electronics 420C performs a predetermined sequence of signal processing on the input from the 2D transducer 41. In one example, the 2D transducer 41 has 24 azimuth elements and 24 elevation elements. The exemplary 2D transducer 41 utilizes 576 receive elements and outputs the same number of channel signals. In certain situation, the 2D transducer 41 optionally utilizes less than all of the available receive elements. Thus, the receive electronics 420C receives the N-channel signals from the 2D transducer 41 to via connections as indicated by arrows. In this exemplary embodiment, the receive electronics 420C includes a low noise amplifer (LNA) 421, a cross point switch 426, a voltage gain amplifier 422, an analog-to-digital signal converter (ADC) 424 and beamformer electronics 425. In this particular exemplary embodiment, the low noise amplifier (LNA) 421 initially performs low noise amplification on the N-channel signals directly received from the 2D transducer 41. In other words, there is no switching or any other processing on the N-channel signals between the 2D transducer 41 and the LNA 421. The LNA 421 amplifies signals while it matches input impedance to element impedance for maximum signal-to-noise ratio and bandwidth.

After every one of the signals from the 2D transducer 41 has been pre-processed only by the LNA 421, the cross point switch 426 connects channels together such that the number of output channels is less than the number of input channels to the cross point switch 426. In other words, the cross point switch 426 connects only the pre-processed channel signals in the exemplary embodiment of the current invention. The above connecting or switching operation is reconfigurable in selecting a combination of the reduced number of channels and the positions of the corresponding receiving elements in the transducer array for the preprocessed channel signals. As a result of the switching in the receive electronics 420C, some of the original signals from the 576 received elements are now combined into the 24 channel signals, which is the predetermined ratio of 576:24. The above connecting or switching operation is performed for optimizing signal fidelity along an ultrasound beam direction of interest.

After the switching process, the receive electronics 420C of the exemplary embodiment further performs additional signal processing within a probe handle 300. The 24 channel signals now are now inputted into the voltage gain amplifier (VGA) 422, which performs voltage gain amplification on the 24 channel signals in this particular exemplary embodiment. The VGA 422 amplifies signals while it gains changes with time and or depth. Subsequently, the analog-to-digital signal converter (ADC) 424 performs analog-to-digital signal conversion on the output from the VGA 422. A resolution level of the analog-to-digital conversion is related to a ratio between a reduced number of output channels and an original number of input channels in the connecting or switching operation. After the beamformer electronics 425 receives the converted digital signal from the ADC 424, the beamformer electronics 425 delays and sums the digital signals in order to form a coherent ultrasound beam.

In above exemplary embodiment, the receive electronics 420C includes the low noise amplifer (LNA) 421, the cross point switch 426, the voltage gain amplifier (VGA) 422, the analog-to-digital signal converter (ADC) 424 and the beamformer electronics 425. Since these components are illustrative, other embodiments are not necessarily limited to a combination of the components and a particular sequence of the components in order to practice the current invention. Furthermore, other alternative embodiments are not necessarily limited to a combination of the components that are located in the probe in order to practice the current invention.

Still referring to FIG. 6, the third embodiment of the probe 300 also includes the transmit electronics 430A, which in turn further includes a transmit pulser 431. As an arrow from the transmit electronics 430A indicates, the transmit pulser 431 controls the generation of ultrasound pulses from transmit elements through a predetermined number of N channels in the 2D transducer 41. In the exemplary embodiment of the probe 300, the transmit electronics 430A performs substantially no preprocessing that is equivalent or related to the above discussed preprocessing of the receive electronics 420C.

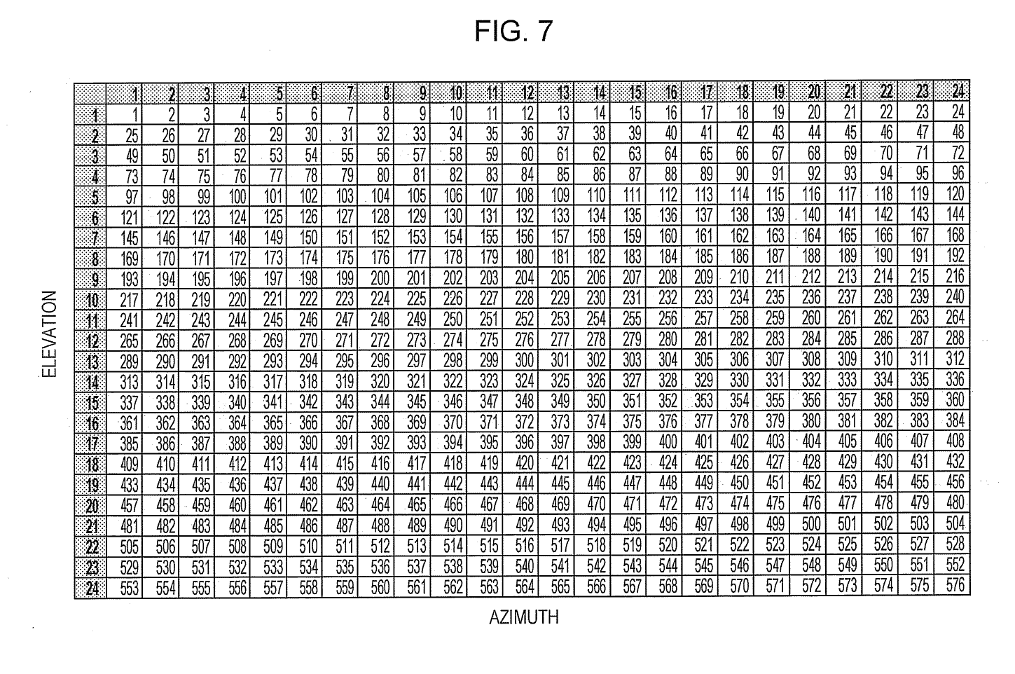

FIG. 7 is a diagram illustrating one embodiment of a two-dimensional array of transducer elements in the probe according to the current invention. The illustrative embodiment has two-dimensional array transducer elements that are electronically configured into rows and columns. In this exemplary transducer array, the rows and columns of transducer elements are respectively organized into 24 elements in Elevations and Azimuth. Each element is numbered from 1 through 576 in the example. The array is electronically configured to treat all of the elements independently as a traditional 2D array. In this way, the array can provide 3D/4D image data. For example, a user sees real-time 2D images of a region of interest such as an organ in a patient so that a user places the probe more accurately with respect to the organ. In this way, the user optionally initiates 2D and or 3D/4D imaging data based upon the visual confirmation of a predetermined target as he or she examines the display monitor in a predetermined survey mode. To implement the above process, the dedicated groups of the transducer elements are efficiently implemented in the probe according to the current invention.

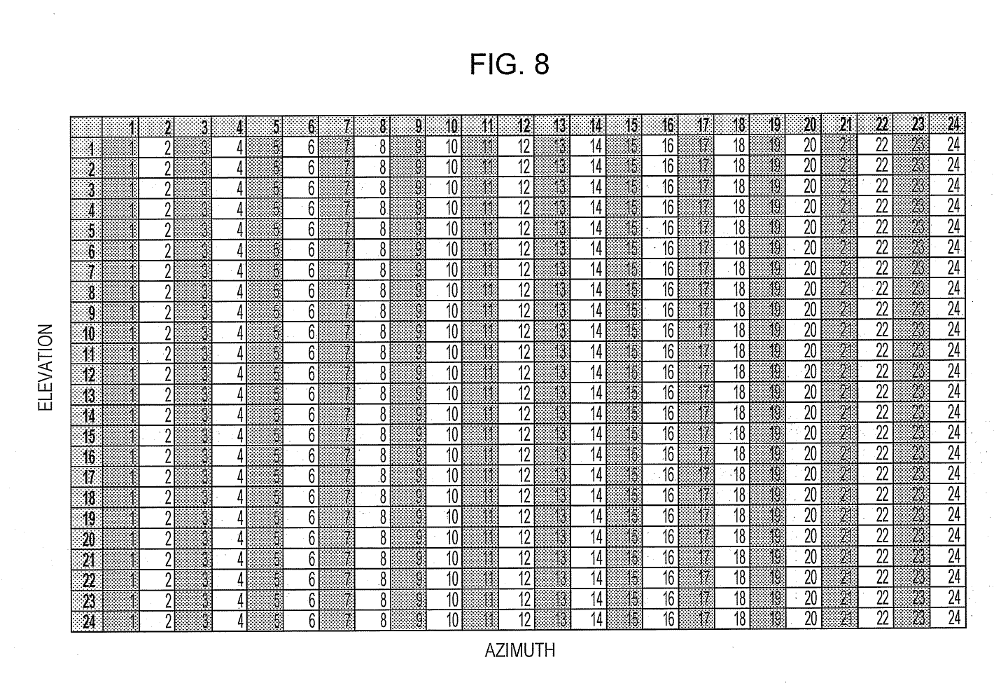

Now referring to FIG. 8, a diagram illustrates a first exemplary pattern of a two-dimensional array of transducer elements in a predetermined survey mode according to the current invention. The diagram illustrates one exemplary switch or multiplexer connection pattern. That is, when the switch or multiplexer is activated, the preprocessed image signals are connected from the receive element channels in the elevation direction so the connected channels originate from columns of receive elements. That is, the 576 input channels of FIG. 7 are reduced to 24 output channels as shown in FIG. 8. This exemplary array pattern diagram does not imply that the receive element channels in the elevation direction are only used in the array. Quite contrarily, unless other wise indicated, all of the receive elements generally receive the ultrasound echoes, and all of these image signals undergo a predetermined set of preprocessing before the preprocessed image signals are connected by a connection unit such as a switch or a multiplexer. By the same token, unless other wise indicated, all of the transmit elements transmit ultrasound pulses.

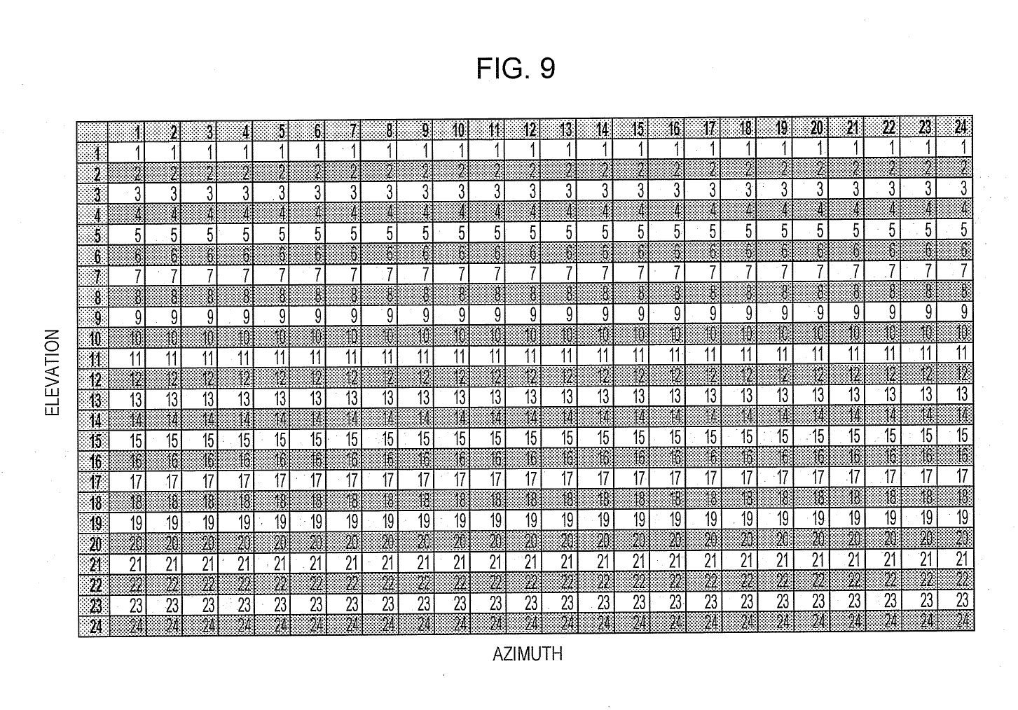

FIG. 9 is a diagram illustrating a second exemplary pattern of a two-dimensional array of transducer elements in a predetermined survey mode according to the current invention. The diagram illustrates another exemplary switch or multiplexer connection pattern. That is, when the switch or multiplexer is activated, the preprocessed image signals are connected from the receive element channels in the Azimuth direction so the connected channels originate only from rows of receive elements. That is, the 576 input channels of FIG. 7 are reduced to 24 output channels as shown in FIG. 9. This exemplary array pattern diagram does not imply that the receive element channels in the elevation direction are only used in the array. Quite contrarily, unless other wise indicated, all of the receive elements generally receive the ultrasound echoes, and all of these image signals undergo a predetermined set of preprocessing before the preprocessed image signals are connected by a connection unit such as a switch or a multiplexer. By the same token, unless other wise indicated, all of the transmit elements transmit ultrasound pulses.

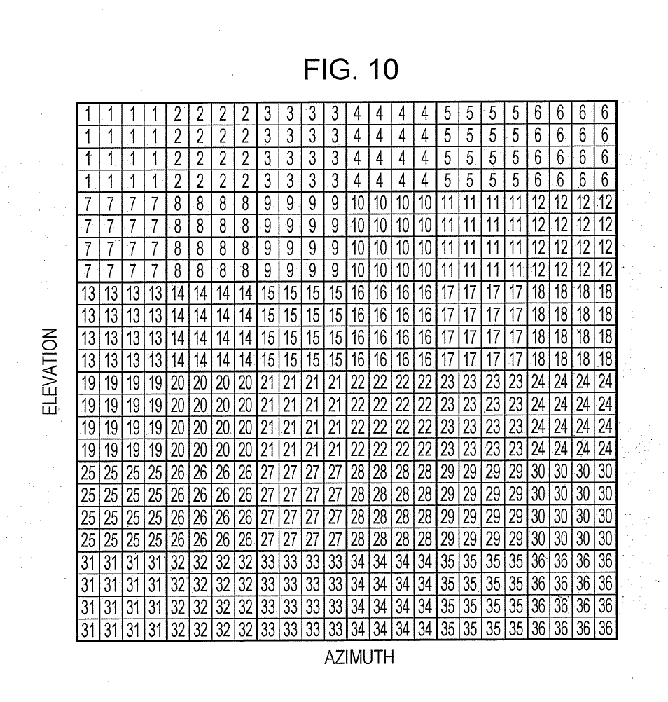

FIG. 10 is a diagram illustrating a fourth exemplary pattern of a two-dimensional array of transducer elements in a predetermined survey mode according to the current invention. The diagram illustrates yet another exemplary switch or multiplexer connection pattern. That is, when the switch or multiplexer is activated, some of the preprocessed image signals are connected together from the receive element channels in the Azimuth-Elevation direction so the connected channels originate only from certain rows and columns of receive elements. The 16 channels in each adjacent four rows and columns of receive elements are connected together in this example. That is, the 576 input channels of FIG. 7 are reduced to 36 output channels as shown in FIG. 10. This exemplary array pattern diagram does not imply that the receive element channels in the elevation direction are only used in the array. Quite contrarily, unless other wise indicated, all of the receive elements generally receive the ultrasound echoes, and all of these image signals undergo a predetermined set of preprocessing before the preprocessed image signals are connected by a connection unit such as a switch or a multiplexer. By the same token, unless other wise indicated, all of the transmit elements transmit ultrasound pulses.

The above described design of the transducer elements is merely illustrative to practice the claimed invention. Although the specification does not elaborate other variations, various configurations are optionally included in grouping the receive elements and connecting their channel inputs based upon the ordinary skill in the art in order to practice the claimed invention.

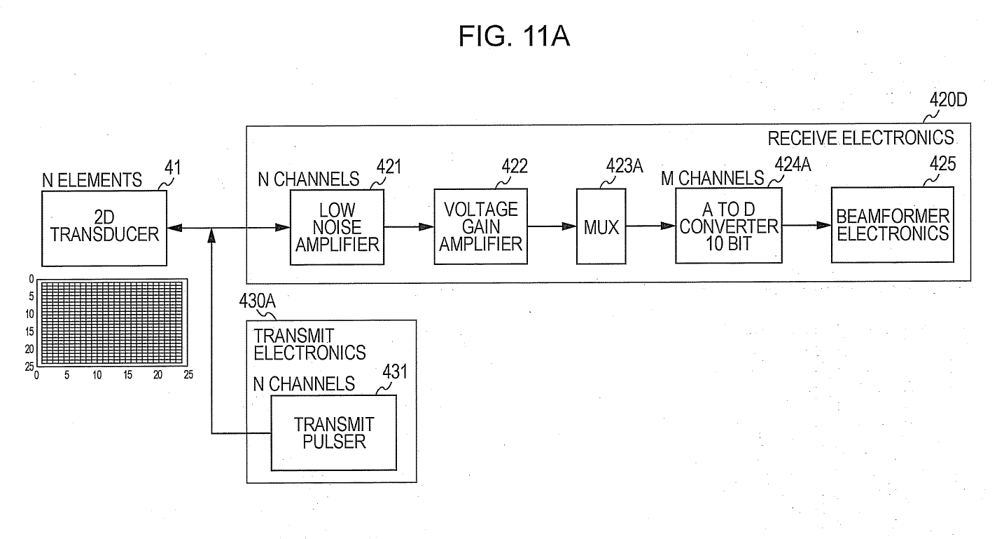

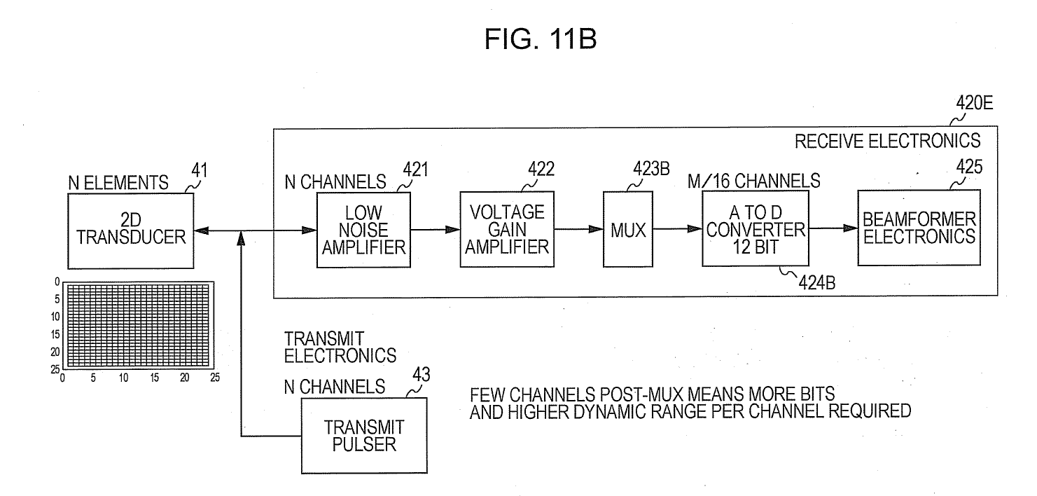

Now referring to FIGS. 11A and 11B, each of the two diagrams illustrates further detail of the first embodiment of the probe according to the current invention. The first embodiment of the probe 100 includes a 2D transducer 41, the transmit electronics 430A and a receive electronics 420D or 420E, which is a particular example of the receive electronics 420 of FIG. 3. The 2D transducer 41 is connected to both the transmit electronics 430A and the receive electronics 420D or 420E so that the 2D transducer 41 transmits ultrasound pulses from transmit elements towards a region of interest of a subject and receive ultrasound echoes that have been reflected from the region of interest of the subject at a first predetermined number of receiving elements of the transducer array so that the ultrasound echoes are converted into the first predetermined number of channel signals.

Still referring to FIGS. 11A and 11B, the receive electronics 420D and 420E perform a predetermined sequence of signal processing on the input from the 2D transducer 41. The 2D transducer 41 utilizes a predetermined number of N receive elements, which output N number of channel signals. In certain situation, the 2D transducer 41 optionally utilizes less than all of the available receive elements. Thus, the receive electronics 420D and 420E receive the N-channel signals from the 2D transducer 41 to via connections as indicated by arrows. In these exemplary embodiments, the receive electronics 420D and 420E both include a low noise amplifier (LNA) 421, a voltage gain amplifier 422 and beamformer electronics 425. In these particular exemplary embodiments, the low noise amplifier (LNA) 421 initially performs low noise amplification on the N-channel signals directly received from the 2D transducer 41. In other words, there is no switching or any other processing on the N-channel signals between the 2D transducer 41 and the LNA 421. The LNA 421 amplifies signals while it matches input impedance to element impedance for maximum signal-to-noise ratio and bandwidth. Subsequently, the voltage gain amplifier (VGA) 422 performs voltage gain amplification on the output from the LNA 421 in these particular exemplary embodiments. The VGA 422 amplifies signals while it gains changes with time and or depth.

Now referring only to FIG. 11A, after every one of the signals from the 2D transducer 41 has been pre-processed by the LNA 421 and the VGA 422, a multiplexer (MUX) 423A connects channels together such that the number of output channels is less than the number of input channels to the MUX 423A. In other words, the MUX 423A multiplexes only the pre-processed channel signals in the exemplary embodiment of the current invention. The above connecting or switching operation is reconfigurable in selecting a combination of the reduced number of channels and the positions of the corresponding receiving elements in the transducer array for the preprocessed channel signals. As a result of the multiplexing in the receive electronics 420D, some of the original signals from the N received elements are now combined into the M channel signals, which is less than the predetermined number N. The above connecting or switching operation is performed for optimizing signal fidelity along an ultrasound beam direction of interest. Although the exemplary embodiment utilizes a multiplexer, another type of switching unit such as a cross point switch is optionally utilized to practice the current invention.

Still referring to FIG. 11A, after multiplexing process, the receive electronics 420D of the exemplary embodiment further performs additional signal processing within a probe handle 100. The multiplexed M channel signal now undergoes analog-to-digital signal conversion at a 10-bit analog-to-digital signal converter (ADC) 424A. A resolution level of the analog-to-digital conversion is related to a ratio between a reduced number of output channels and an original number of input channels in the connecting or switching operation. In this particular exemplary embodiment, the 10-bit analog-to-digital signal converter (ADC) 424A is proper for the M/N ratio. After the beamformer electronics 425 receives the converted digital signal from the 10-bit ADC 424A, the beamformer electronics 425 delays and sums the digital signals in order to form a coherent ultrasound beam. In the exemplary embodiment of the probe 100, the transmit electronics 430A performs substantially no preprocessing that is equivalent or related to the above discussed preprocessing of the receive electronics 420D.

In comparison to the receive electronics 420D of FIG. 11A, the receive electronics 420E of FIG. 11B performs substantially the same functions except for the difference in a multiplexer (MUX) 423B and an analog-to-digital signal converter (ADC) 424B. Now referring only to FIG. 11B, after every one of the signals from the 2D transducer 41 has been pre-processed by the LNA 421 and the VGA 422, the MUX 423B connects channels together such that the number of output channels is less than the number of input channels to the MUX 423B. The above connecting or switching operation is reconfigurable in selecting a combination of the reduced number of channels and the positions of the corresponding receiving elements in the transducer array for the preprocessed channel signals. As a result of the multiplexing in the receive electronics 420E, some of the original signals from the N received elements are now combined into the M/16 channel signals, which is less than the predetermined number N. In other words, the MUX 423B of the receive electronics 420E of FIG. 11B connects the input channels to a smaller number of channels than the MUX 423A of the receive electronics 420D of FIG. 11A.

Still referring to FIG. 11B, after multiplexing process, the receive electronics 420E of the exemplary embodiment further performs additional signal processing within a probe handle 100. The multiplexed M/16 channel signal now undergoes analog-to-digital signal conversion at a 12-bit analog-to-digital signal converter (ADC) 424B. As already described, the resolution level of the analog-to-digital conversion is related to a ratio between a reduced number of output channels and an original number of input channels in the connecting or switching operation. In this particular exemplary embodiment, the 12-bit ADC 424B is provided for the M/16 to N ratio. The reduction in channel count allows power to be allocated for enabling a higher channel dynamic range. In the above illustrations in FIGS. 11A and 11B, the ADC bit count is increased from 10 bits to 12 bits.

After the beamformer electronics 425 receives the converted digital signal from the 12-bit ADC 424B, the beamformer electronics 425 delays and sums the digital signals in order to form a coherent ultrasound beam. In the exemplary embodiment of the probe 100, the transmit electronics 430A performs substantially no preprocessing that is equivalent or related to the above discussed preprocessing of the receive electronics 420E.

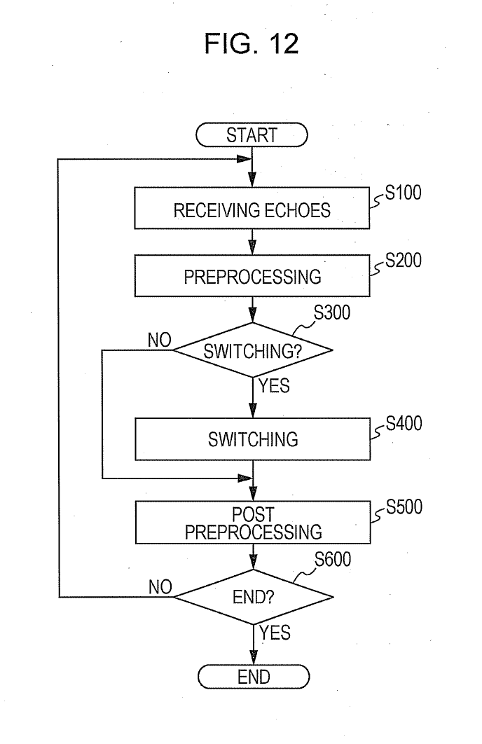

FIG. 12 is a flow chart illustrating the steps or acts involved in one embodied process of operation with respect to the use of the probe according to the current invention. The embodied process of receive operation in the probe includes a step S100 of receiving ultrasound echoes at receive elements in the array. For example, after a transducer array outputs N number of channel signals from a predetermined number of N receive elements, the receive electronics receives the N-channel signals from the transducer array.

In a step S200, the received signals are preprocessed. The preprocessing step S200 is accomplished by a low noise amplifier (LNA) and or a voltage gain amplifier (VGA). For example, the LNA initially performs low noise amplification on the N-channel signals directly received from the transducer. In other words, there is no switching or any other processing on the N-channel signals between the transducer array and the LNA. The LNA amplifies signals while it matches input impedance to element impedance for maximum signal-to-noise ratio and bandwidth. Subsequently, the VGA performs voltage gain amplification on the output from the LNA in this particular exemplary process. The VGA amplifies signals while it gains changes with time and or depth. Although the above exemplary preprocessing step involves a combination of low noise amplification and voltage gain amplification, the preprocessing step S200 is not limited to this particular combination and is optionally accomplished by any other relevant combination of preprocessing operations. The above described preprocessing operation improves the image quality of the input signal.

After every one of the signals from the transducer array has been pre-processed by the LNA and or the VGA in the step S200, it is determined whether or not a switching step takes place in a step S300. It is determined in the step S300 whether or not a switching or connecting operation should take place. If it is determined that a switching or connecting operation should take place in the step S300, the exemplary process proceeds to a step S400. On the other hand, if it is determined that a switching or connecting operation should not take place in the step S300, the exemplary process proceeds to the step S500 to avoid the switching or connecting operation.

In the switching step S400, the preprocessed channel signals are now combined by connecting a predetermined set of channels together so as to output a fewer number of channel signals. For example, the multiplexer (MUX) connects channels together such that the number of output channels is less than the number of input channels to the MUX. In other words, the MUX multiplexes only the pre-processed channel signals in the exemplary process of the current invention. The above connecting or switching step S400 is reconfigurable in selecting a combination of the reduced number of channels and the positions of the corresponding receiving elements in the transducer array for the preprocessed channel signals. The above connecting or switching operation is performed for optimizing signal fidelity along an ultrasound beam direction of interest. Although the exemplary embodiment utilizes a multiplexer, another type of switching unit such as a cross point switch is optionally utilized to practice the current invention.

After the switching step S400, additional signal processing is performed inside or outside of a probe handle in a post processing step S500. For example, the multiplexed M channel signal undergoes analog-to-digital signal conversion using an analog-to-digital signal converter (ADC) and delay/summation using the beamformer electronics. A resolution level of the analog-to-digital conversion is related to a ratio between a reduced number of output channels and an original number of input channels in the connecting or switching operation. After the beamformer electronics receives the converted digital signal from the ADC, the beamformer electronics delays and sums the digital signals in order to form a coherent ultrasound beam. The post processing step S500 is not limited to the above described operation.

After the post processing step S600, it is further determined whether or not the embodiment process should terminate in a step S600. If it is determined that the embodiment process should terminate in the step S600, the embodiment process proceeds to an end. On the other hand, if it is determined that the embodiment process should continue in the step S600, the embodiment process proceeds to the step S100 to the above described steps.

According to any and all embodiments explained above, advantages include an improved or efficient workflow of the ultrasound examination based upon the improved image in a survey mode. Similar to other modalities such as X-ray, computer tomography (CT) or magnetic resonance imaging (MRI), the improved workflow involves a process where patients are examined using an ultrasound imaging device as quickly as possible to acquire data and the stored diagnostic images are manipulated and analyzed typically after the patients have already left the examination sessions. Particularly, in the advent of volumetric imaging or also known as sonographic tomography, a single sweep of a region using modem 2D array ultrasound may provide all necessary data for later analyses. To ensure that the transducer is positioned to capture a diagnostically relevant volume and that the system parameters are appropriately set for meaningful data, and to reduce power, a conventional 2D image is optionally provided as a good acoustic window to the operator before initiating the diagnostically relevant 3D/4D data. Ultimately, the improved workflow enhances the utilization of the imaging devices.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope of the inventions.

What is claimed is:

1. A method of ultrasound imaging, comprising the steps of:
transmitting ultrasound pulses from transmit elements of a transducer array towards a region of interest of a subject;
receiving ultrasound echoes that have been reflected from the region of interest of the subject at a first predetermined number of receiving elements of the transducer array, the ultrasound echoes being converted into the first predetermined number of channel signals;
preprocessing the channel signals to generate preprocessed channel signals; and
connecting the preprocessed channel signals into a second predetermined number of output channel signals, the second predetermined number being smaller than the first predetermined number.

2. The method of ultrasound imaging according to claim 1 wherein the first predetermined number is all of the receiving elements of the transducer array.

3. The method of ultrasound imaging according to claim 1 wherein the first predetermined number is less than all of the receiving elements of the transducer array.

4. The method of ultrasound imaging according to claim 1 wherein said connecting step is reconfigurable in selecting a combination of the second predetermined number and positions of the receiving elements in the transducer array for the preprocessed channel signals.

5. The method of ultrasound imaging according to claim 1 wherein said preprocessing step further comprises a step of performing low noise amplification on the channel signals.

6. The method of ultrasound imaging according to claim 1 wherein said preprocessing step further comprises a step of performing voltage gain amplification on the channel signals.

7. The method of ultrasound imaging according to claim 1 wherein said preprocessing step further comprises a step of performing in any sequence low noise amplification and voltage gain amplification on the channel signals.

8. The method of ultrasound imaging according to claim 1 further comprising a combination of additional steps of:
converting the output channel signals into digital signals; and
forming a coherent ultrasound beam based upon the digital signals.

9. The method of ultrasound imaging according to claim 8 wherein a resolution level of said converting step is related to a ratio between the second predetermined number and the first predetermined number.

10. The method of ultrasound imaging according to claim 1 wherein said connecting step is performed for optimizing signal fidelity along an ultrasound beam direction of interest.

11. A system for ultrasound imaging, comprising:
a transducer array for transmitting ultrasound pulses from transmit elements towards a region of interest of a subject, said transducer array also receiving ultrasound echoes that have been reflected from the region of interest of the subject at a first predetermined number of receiving elements of the transducer array, the ultrasound echoes being converted into the first predetermined number of channel signals;
a preprocessing unit connected to said receiving elements for preprocessing the channel signals to generate preprocessed channel signals; and
a switch unit connected to said preprocessing unit for connecting the preprocessed channel signals into a second predetermined number of output channel signals, the second predetermined number being smaller than the first predetermined number.

12. The system for ultrasound imaging according to claim 11 wherein the first predetermined number is all of said receiving elements of said transducer array.

13. The system for ultrasound imaging according to claim 11 wherein the first predetermined number is less than all of said receiving elements of said transducer array.

14. The system for ultrasound imaging according to claim 11 wherein said switch unit is reconfigurable in selecting a combination of the second predetermined number and positions of said receiving elements in said transducer array for the preprocessed channel signals.

15. The system for ultrasound imaging according to claim 11 wherein said preprocessing unit further comprises a low noise amplifier for performing low noise amplification on the channel signals.

16. The system for ultrasound imaging according to claim 11 wherein said preprocessing unit further comprises a voltage gain amplifier for performing voltage gain amplification on the channel signals.

17. The system for ultrasound imaging according to claim 11 wherein said preprocessing unit performing in any sequence low noise amplification and voltage gain amplification on the channel signals.

18. The system for ultrasound imaging according to claim 11 further comprising:
an analog-to-digital converter connected to said switch unit for converting the output channel signals into digital signals; and
a beamforming unit connected to said analog-to-digital converter for forming a coherent ultrasound beam based upon the digital signals.

19. The system for ultrasound imaging according to claim 18 wherein a dynamic range of said analog-to-digital converter is related to a ratio between the second predetermined number and the first predetermined number.

20. The system for ultrasound imaging according to claim 11 wherein said switch unit optimizes signal fidelity along an ultrasound beam direction of interest.

21. A system for ultrasound imaging, comprising:
a transducer array for transmitting ultrasound pulses from transmit elements towards a region of interest of a subject, said transducer array also receiving ultrasound echoes that have been reflected from the region of interest of the subject at a first predetermined number of receiving elements of the transducer array, the ultrasound echoes being converted into the first predetermined number of channel signals;
a preprocessing unit connected to said receiving elements for preprocessing the channel signals to generate preprocessed channel signals;
a switch unit connected to said preprocessing unit for connecting the preprocessed channel signals into a second predetermined number of output channel signals, the second predetermined number being smaller than the first predetermined number; and
an analog-to-digital converter connected to said switch unit for converting the output channel signals into digital signals, a dynamic range of said analog-to-digital converter being related to a ratio between the second predetermined number and the first predetermined number.

22. A system for ultrasound imaging, comprising:
a transducer array for transmitting ultrasound pulses from transmit elements towards a region of interest of a subject, said transducer array also receiving ultrasound echoes that have been reflected from the region of interest of the subject at a first predetermined number of receiving elements of the transducer array, the ultrasound echoes being converted into the first predetermined number of channel signals;
a switch unit connected to said transducer array for connecting the channel signals into a second predetermined number of output channel signals, the second predetermined number being smaller than the first predetermined number; and
an analog-to-digital converter connected to said switch unit for converting the output channel signals into digital signals, a dynamic range of said analog-to-digital converter being related to a ratio between the second predetermined number and the first predetermined number.

23. A method of ultrasound imaging, comprising the steps of:
transmitting ultrasound pulses from transmit elements of a transducer array towards a region of interest of a subject;
receiving ultrasound echoes that have been reflected from the region of interest of the subject at a first predetermined number of receiving elements of the transducer array, the ultrasound echoes being converted into the first predetermined number of channel signals;
connecting the channel signals into a second predetermined number of output channel signals, the second predetermined number being smaller than the first predetermined number; and
converting the output channel signals into digital signals, a dynamic range of said analog-to-digital converter being related to a ratio between the second predetermined number and the first predetermined number.
